(19) United States
(12) Patent Application Publication
(10) Pub. No.: US 2018/0294056 A1
(43) Pub. Date: Oct. 11, 2018

(54) COMPUTER-IMPLEMENTED METHOD FOR OPTIMIZING THE USAGE OF MEDICAL IMAGING APPARATUSES

(71) Applicant: Siemens Healthcare GmbH, Erlangen (DE)

(72) Inventors: Eva Rothgang, Schwaig bei Nuernberg (DE); Matthias Siegert, Bamberg (DE)

(73) Assignee: Siemens Healthcare GmbH, Erlangen (DE)

(21) Appl. No.: 15/946,854

(22) Filed: Apr. 6, 2018

(30) Foreign Application Priority Data
Apr. 7, 2017 (DE) 102017206029.8

(51) Int. Cl. G16H 40/40; G06Q 10/10; G16H 40/20

(52) U.S. Cl. CPC G16H 40/40; G16H 40/20; G06Q 10/1095

(57) ABSTRACT

A method for optimizing operation of at least one medical imaging apparatus, and a computer for implementing such a method, an appointment request from a patient, and at least one patient attribute, are entered into a computer, and the patient attribute is forwarded from the computer to the medical imaging apparatus. The medical imaging apparatus provides the computer with the duration of the medical imaging procedure to be conducted, as a function of the patient attribute, and forwards a designation of that duration to the computer. The computer selects an appointment dependent on the duration and the availability of the medical imaging apparatus. The selected appointment is stored by the computer.

### BACKGROUND OF THE INVENTION

### Field of the Invention

The present invention relates to a method for optimizing the usage of medical imaging apparatuses, a method for operating an appointments scheduling system, a method for operating a medical imaging apparatus, an interface, in particular for minimizing changeover times, and a non-transitory data medium and a medical imaging apparatus that implement such a method.

### Description of the Prior Art

Currently, operation of medical imaging apparatuses typically entails performing examinations lasting a predefined period of time, e.g. 30 minutes per patient. The duration of the medical imaging process is not adjusted to the patient or to the medical imaging method that is to be performed. Furthermore, no feedback takes place between the appointments scheduling system and the medical imaging apparatus. As a result, no adjustment of the planned duration of the examination to the actual examination time takes place. This leads to a significantly lower utilization of the medical imaging apparatus, since the examinations in the appointments scheduling system do not correspond to reality. This leads in particular to increased waiting times for the patients, as well as to a significantly lower utilization of the medical imaging apparatus.

### SUMMARY OF THE INVENTION

An object of the invention is to resolve the known shortcomings as well as to create an added value, in particular by an increase in the utilization of the medical imaging apparatus, as well as a significant reduction in the waiting times for a patient.

According to the invention, a computer-implemented method for optimizing the operation of medical imaging apparatuses in one or more service centers has the following steps.

A patient makes an appointment request an appointments scheduling system.

At least one patient attribute is acquired by the appointments scheduling system.

The patient attribute is forwarded from the appointments scheduling system to the medical imaging apparatus.

The duration of a medical imaging process is provided by the medical imaging apparatus as a function of the patient attribute, and this duration is forwarded to the appointments scheduling system.

An appointment is selected by the appointments scheduling system as a function of the duration of the medical imaging process and availability of the medical imaging apparatus(s).

The appointment selected by the patient is stored by the appointments scheduling system.

A service center, in the present context, is an institution that has at least one medical imaging apparatus or a number of medical imaging apparatuses and has the necessary specialist staff qualified to perform the imaging procedures. An appointments scheduling system can be a PC, a call center and/or an online server, the appointments scheduling system being configured to receive an appointment request submitted by a patient. In particular, the appointments scheduling system includes an electronic calendar having an interface to patients, e.g. online or via an operator. An appointment request by a patient can be an appointment request made by a telephone call, an online form or an internet website. The acquisition of at least one patient attribute can be accomplished by a telephone call between the appointments scheduling system and the patient, via an online form or via an internet website, wherein the acquisition entails storing the patient attribute in the appointments scheduling system.

A patient attribute can relate to a variety of characteristics, e.g. the name of a patient, a type of scan that the patient wishes to undergo, information with regard to implants present in the patient, or information with respect to personal details provided by a patient.

Communicating a patient attribute from the appointments scheduling system to the medical imaging apparatus involves the sending of data packets between the two systems. In this process, the data packets preferably are encrypted.

Particularly preferably, the duration of a medical imaging procedure as a function of a patient attribute is provided by the linking of the patient attribute with the type of medical imaging procedure that is to be performed on the patient, in combination with the type of acquisition coil that is installed in the medical imaging apparatus at the time of the appointment request. The selection of an appointment by the appointments scheduling system is preferably carried out as a function of the duration of the medical imaging process and an availability of the medical imaging apparatus on which the medical imaging procedure is to be performed, and consequently it is sought to combine the same or similar imaging procedures having appointments as close as possible to one another or adjacent in time. Once an appointment has been successfully selected, the appointments scheduling system stores all the information, in particular the date of the appointment, the patient attributes and the availability of the medical imaging apparatus, in the appointments scheduling system.

An advantage of this embodiment is that the number of acquisition coil changes is reduced to a minimum as a result, there is a higher utilization of the medical imaging apparatus, and a higher level of customer satisfaction is achieved thanks to shorter waiting times. The amount of effort required on the part of specialist medical staff for issuing appointments is also significantly reduced. An alternative embodiment, such as the consolidation of the appointments scheduling system and the medical imaging apparatus in an integrated system, is conceivable, in which case the computer-implemented method for optimizing operation can be performed on the integrated system. Equally conceivable is an alternative embodiment in which the appointment request and the appointments scheduling system are handled by a single person with the use of a telephone. A preferred embodiment is that an appointment request is submitted via an internet website, wherein the website acquires at least one patient attribute, for example the name, the type of scan or the imaging process that is to be performed, and the appointments scheduling system forwards all of the information to a medical imaging apparatus and, as response to the appointment request, makes an appointment slot available to the patient, who can either accept the appointment or select another appointment.

As an alternative there is also a step wherein the selected appointment is provided as an output on an interface of the appointments scheduling system. The interface can be a point of access to various other software systems or a point of access to an internet- or cloud-based appointments scheduling system. The advantage of this embodiment is the potential high degree of integration into existing computer systems, as well as a combination with other software systems in order to achieve a further improvement in the operation of a medical imaging apparatus.

Alternatively, the method for optimizing the operation of the medical imaging apparatuses can be performed.

Particularly advantageously, the steps after the patient attribute is acquired are performed automatically. This advantageously leads to a reduction in the potential for error as well as to a reduction in the process time for assigning appointments.

According to one embodiment, the method has the following steps. The estimated duration of an ongoing medical imaging process is determined during the ongoing medical imaging process. The estimated duration of the ongoing medical imaging process is forwarded to the appointments scheduling system and the estimated duration is compared with its provided duration. If necessary, a data packet is provided containing the estimated end time. If necessary, adjustment of the following appointments is implemented by the appointments scheduling system.

Determining the estimated duration during the ongoing medical imaging process preferably entails registering the current progress of the medical imaging process, which is preferably recorded in hours or minutes or seconds. A forwarding of the estimated duration, for example by means of an electronic or wireless communication, preferably takes place by means of the appointments scheduling system. The appointments scheduling system preferably makes a comparison between the estimated duration of the medical imaging process and the provided duration of the medical imaging process, which was specified at the time the appointment was assigned. In the comparison, a difference between the estimated duration and the provided duration is determined. With the use of this difference, an end time of the medical imaging process is calculated. If this difference exceeds a threshold value, for example five minutes, this end time is communicated to the appointments scheduling system by means of a data packet. The appointments scheduling system is able to adjust subsequent appointments as a function of the end time of the medical imaging process. The adjustment can comprise contacting the patients occupying the subsequent appointment slots. This embodiment has advantages of a significant reduction in patient waiting times, since patients are informed in good time about a deferred or delayed appointment, and an increase in the utilization of the medical imaging apparatus as a result of greater patient punctuality as well as greater patient satisfaction, since no unnecessary waiting times occur. For example, a medical imaging process is performed such that the estimated duration or the estimated end of the medical imaging process is determined during the current medical imaging process and this information is forwarded to the appointments scheduling system, whereupon the appointments scheduling system compares the estimated end with the scheduled end, as a result of which the appointments scheduling system, if necessary, sends a message by SMS or email to the patient occupying the following appointment slot, for example a notification informing the patient that the appointment will be delayed by 15 minutes.

The method has the following further steps:
providing a setup time as a function of the patient attribute to the appointments scheduling system by the medical imaging apparatus,
taking the setup time into consideration in the appointment selection by the appointments scheduling system.

Providing a setup time as a function of a patient attribute refers to a setup time or an allocated time for the medical imaging apparatus that comes about for example as a result of a need to change an acquisition coil of the medical imaging apparatus owing to the fact that a different medical imaging method is to be performed on the patient about to be examined from that conducted on the previous patient. When the setup time is taken into consideration during the appointment selection, patients undergoing the same medical imaging processes are preferably examined one after another with the aid of the medical imaging apparatus. The advantage of this embodiment is a more efficient assignment of appointments, since the number of acquisition coil changes is significantly reduced and the workload of the specialist medical staff is lightened. At the time of an appointment request, it can be determined for example by means of the patient attribute whether it is necessary to reconfigure the medical imaging apparatus setup or not, and, if so, how long the setup time will be.

Preferably, an appointment is selected such that ideally no setup times occur, in that as far as possible successive appointment slots are selected for medical imaging processes having the same patient attribute. Preferably, the same patient attribute is the same body region, or the same scanning position on the medical imaging apparatus, in order thereby to minimize possible setup times. Identical patient attributes are preferably handled in sequence and in this way the operation of the medical imaging apparatus is organized more efficiently. For example, the same patient attributes can be applied using the same medical imaging process on two successive appointments or else also successively with a time delay, for example such that the same medical imaging processes are performed on two consecutive days. The advantage of this embodiment is a reduction in the setup times as well as a less intensive utilization of the acquisition coils and a reduction in the changeover times of the installations and a resulting reduction in the workload of the specialist medical staff.

In another embodiment of the invention, the method is performed such that the duration of the medical imaging process is determined or calculated with the aid of averaged durations from medical imaging processes performed on the medical imaging apparatus over a period of time, preferably between one day and three months. Averaged durations of the medical imaging process are determined for the same medical imaging processes. In the present exemplary embodiment, this means that shoulder scans, for example, are analyzed in terms of their duration and an average value is formed therefrom. Said durations are collected over a time period of preferably between one day and three months and averaged, and subsequently used for providing the duration of the medical imaging process. This leads to an improvement in the prediction accuracy of the duration of the medical imaging process, as well as to an automatic adjustment to the local conditions or to the efficiency of the specialist medical staff on site.

The method can beneficially be performed, wherein the patient attribute may be a name of the patient and/or a type of imaging process and/or a part of the body and/or an associated acquisition coil and/or one or more implants present in the patient and/or an indication and/or personal details provided by the patient. The name of a patient that is used may be, for example, a first given name and family name as well as the title of the patient. The type of imaging process may be for example the composition formed from medical imaging apparatus as well as acquisition direction and frequency. Preferably, a patient attribute describes the part of the body or the body region that is to be examined by means of the medical imaging process. Depending on the type of medical imaging process and the part of the body, the associated acquisition coil for example can be defined as a patient attribute. Preferably, one or more implants of the patient may for example also be noted as a patient attribute. In addition to the number of implants of the patient, the type of implants may also be recorded, for example. Particularly preferably, a patient attribute is an indication, a pre-assessment for diagnostic purposes having already been made by specialist medical staff. A patient attribute may equally be for example personal details provided by a patient, in which there is contained various information about the patient and the patient's declaration of informed consent to a medical imaging session. Particularly advantageous in this embodiment is the avoidance of unnecessary premature terminations of a medical imaging process, because a comprehensive investigation with regard to implants present in the patient has already taken place. Similarly, changeover times for swapping acquisition coils are reduced to a minimum because the patient has been allocated an appointment slot on the basis of the necessary medical imaging process. In an exemplary embodiment, the patient specifies various patient attributes in an internet form, such as his/her name, for example, the type of medical imaging process, for example computed tomography, the part of the body that is to be examined, for example the shoulder of the patient, and information about the presence of one or more implants in the patient, as well as personal details of the patient.

In a preferred embodiment, the method can be performed, wherein a patient may decline an appointment and reports this to the appointments scheduling system, wherein the appointments scheduling system generates at least one further appointment by repeating the appointment selecting step, wherein the patient receives the further appointment and sends the appointments scheduling system an acknowledgment, wherein the step of storing is performed if the acknowledgment is positive. In this situation, declining the appointment preferably means that the patient reports to the appointments scheduling system that he/she is unable to keep the proposed appointment. This causes the appointments scheduling system to initiate the generation of a further appointment, wherein at least one appointment, preferably a number of appointments, is or are generated by single or multiple iteration of the step of selecting the appointment. Preferably, the patient receives at least one or more further appointments, wherein he/she reports back to the appointments scheduling system to confirm an appointment that he/she wishes to accept. Following the positive acknowledgment, storing step is performed and the scheduled appointment is stored. The advantage of this embodiment is the increased utilization of the medical imaging apparatus, as well as a reduction in patient no-shows and an increase in customer satisfaction owing to the availability of an acceptable alternative appointment. As an example, a patient can decline an appointment with the aid of an online form, as a result of which the appointments scheduling system proposes at least one further appointment to the patient. The patient can decide whether or not to accept this appointment. As soon as an appointment has been selected by the patient, the step of storing is performed in order to store the corresponding information in the appointments scheduling system.

Beneficially, the method is performed such that the availability of a medical imaging process in the appointment selecting step is determined on the basis of at least one, preferably two or more medical imaging apparatuses, wherein the medical imaging apparatuses may be distributed over a number of service centers. An advantage of this embodiment is an efficient distribution of the patients over a plurality of service centers or medical imaging apparatuses in order in this way to reduce the changeover times to a minimum. The result from this is an increase in the utilization of the medical imaging apparatuses across all service centers. For example, an appointment request is processed simultaneously across a plurality of imaging apparatuses in different service centers and as a result the earliest possible appointment with the shortest possible changeover time is selected for the patient.

According to a further aspect, a method is performed with the steps:
processing of an appointment request of a patient by the appointments scheduling system,
acquisition of at least one patient attribute by the appointments scheduling system,
forwarding an appointment request to a medical imaging apparatus and/or one or more patient attributes to the medical imaging apparatus by the appointments scheduling system,
reception, by the appointments scheduling system, of a response of the medical imaging apparatus, comprising a duration of a medical imaging process and/or an acquisition coil that is installed in the medical imaging apparatus at the time of the appointment request,
calculation, by the appointments scheduling system, of the duration of an appointment on the basis of the appointment request, at least one patient attribute, the duration of the medical imaging process and, if applicable, a length of time for changing the acquisition coil, and
assignment and/or sorting of the appointments on the basis of patient attributes of the patients in order to minimize a number of acquisition coil changes.

Preferably, the appointments scheduling system is configured to process an appointment request of a patient and to acquire at least one patient attribute. Equally preferably, the appointments scheduling system can forward the appointment request and/or one or more patient attributes to a medical imaging apparatus. The appointments scheduling system can receive a response of a medical imaging apparatus, wherein information received by the appointments scheduling system includes a duration of a medical imaging process and an acquisition coil. Preferably, the appointments scheduling system can calculate a duration of the appointment with the use of this information. In order to calculate the duration of an appointment, the appointments scheduling system requires at least one patient attribute, the duration of the medical imaging process and, if applicable, a length of time for changing the acquisition coil. The length of time required for changing the acquisition coil is optional, because this time can be saved if there is no need to change the acquisition coil. The appointments scheduling system can assign and sort appointments on the basis of patient attributes. In so doing, the appointments scheduling system attempts to minimize the number of acquisition coil changes. The advantage of this embodiment is a reduction of acquisition coil changes to a minimum, as well as an increased utilization of the medical imaging apparatus.

According to a further aspect, the invention relates to a method for operating an imaging apparatus, having the following steps:
receiving an appointment request and/or at least one patient attribute,
providing a duration of a medical imaging process as a function of at least one patient attribute,
forwarding the duration of the medical imaging process.

The medical imaging apparatus can receive an appointment request or at least one patient attribute. The receiving is accomplished with the use of data packets which are recorded and interpreted by the medical imaging apparatus. Providing a duration of a medical imaging process on the part of a medical imaging apparatus preferably means that a duration of the medical imaging process is determined on the basis of a patient attribute and said duration is made available. The medical imaging apparatus can forward the determined duration of the medical imaging process. The forwarding is understood as sending information in the form of data packets which a linked device can receive and knows how to interpret. The advantage of this method is that a duration that is appropriate to a patient attribute is determined for the patient attribute and as a result there is no need to employ imprecise default values.

Beneficially, the method is performed, wherein the duration of the medical imaging process is calculated as a function of a patient attribute, said duration comprising a preparation time of the medical imaging process, an acquisition time of the medical imaging process, a post-processing time of the medical imaging process, and, if applicable, a changeover time for swapping an acquisition coil of the medical imaging apparatus, wherein the duration of the medical imaging process is calculated constantly, daily, weekly, quarterly or yearly on the basis of predefined values and/or values determined based on the operation of the medical imaging apparatus and/or on manual inputs on the medical imaging apparatus. As preparation time of the medical imaging process is understood the time required to prepare the medical imaging apparatus for a patient. This includes, for example, cleaning of the medical imaging apparatus or other activities. The acquisition time of a medical imaging process comprises the time required to perform the medical imaging process. By this is understood the time between the start and the end of the medical imaging process. As post-processing time of a medical imaging process is understood for example that the medical imaging apparatus is restored to its original state upon completion of a medical imaging process. The changeover time of an acquisition coil comprises the length of time during which the medical imaging apparatus cannot be used because, for example, the acquisition coil is being changed.

Among the predefined values for calculating the duration of the medical imaging process are values that were stored at the time of shipment of the medical imaging apparatus, which values can be used for calculating the duration of the medical imaging process. Also, the values for calculating the duration of the medical imaging process can preferably be determined during operation of the medical imaging apparatus, wherein the determined values are averaged or smoothed. Equally, the values for calculating the duration of the medical imaging process can be determined by means of a manual input. The duration of the medical imaging processes can preferably be calculated constantly, daily, weekly, quarterly or yearly. This embodiment has the advantage that the length of time required for the medical imaging processes as a function of the patient attributes is adjusted to the local circumstances, and also that a better predictability of the duration is achieved.

In a preferred embodiment, an interface, in particular for minimizing changeover times, between an appointments scheduling system and a medical imaging apparatus has the following features,
the interface is configured to forward an appointment request from the appointments scheduling system to the medical imaging apparatus, wherein the data format can be converted by the interface,
the interface is configured to record a response of the medical imaging apparatus to the appointment request and to pass on said response to the appointments scheduling system, wherein the response can be converted by the interface into a different data format,
the interface of the appointments scheduling system is configured to store the appointment request and/or the response in the event that the appointments scheduling system and/or the medical imaging apparatus cannot be reached.

In other words, the interface serves to forward information between the appointments scheduling system and the medical imaging apparatus and at the same time, if necessary, to convert the data formats of the data packets. If the appointments scheduling system or the medical imaging apparatus is not available, the interface can preferably buffer the information that is currently to be transferred. The advantage of this embodiment is that it enables different software systems or appointments scheduling systems and medical imaging apparatuses from different manufacturers to be networked with one another.

The preferred embodiments are applicable to all aspects or all methods according to the invention.

The invention further relates to a non-transitory data storage medium on which there is stored a program code for performing the method according to the invention. In this case the data medium is preferably embodied as a PC or server hard disk, or as any other type of removable data storage medium.

Finally, the invention is directed to a medical imaging apparatus which is configured to perform the methods for optimizing the operation of medical imaging apparatuses as described above. The medical imaging apparatus can be an MR scanner or a magnetic resonance tomography system or a computed tomography system or a magnetic resonance positron emission tomography system or a computed tomography positron emission tomography system or an X-ray apparatus or a C-arm X-ray apparatus or a mammography system or an ultrasound apparatus or a positron emission tomography system or a SPECT apparatus.

### BRIEF DESCRIPTION OF THE DRAWINGS

FIG. 1 schematically illustrates the method according to the invention for optimizing the operation of medical imaging apparatuses.

FIG. 2: schematically illustrates a medical imaging apparatus and an appointments scheduling system according to the invention.

### DESCRIPTION OF THE PREFERRED EMBODIMENTS

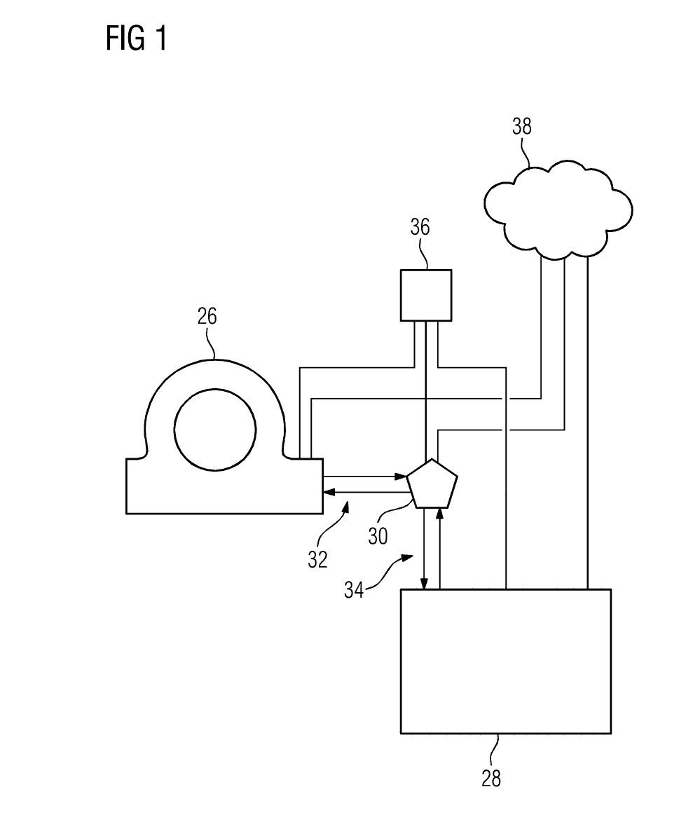

FIG. 1 shows a computer-implemented method for optimizing the operation of medical imaging apparatuses in one or more service centers. The method steps performed within a service center are illustrated in FIG. 1. The method steps can take place in this sequence, although other sequences or combinations of the method steps are also conceivable. The method steps are triggered by an appointment request 10. An acquisition 12 of at least one patient attribute is initiated by the appointment request 10. Once the acquisition 12 has been completed, the next method step follows, namely the forwarding 14 of the acquired patient attribute. In the method step of providing 16 a duration as a function of a patient attribute, the duration of the medical imaging process is determined with the aid of the patient attribute. The providing 16 of the duration is followed by the selection 18 of an appointment as a function of the duration of the medical imaging process. Once an appointment has been successfully selected, it is stored in the method step of storing 20 the appointment. If necessary, the appointment is output at an interface in the method step of outputting 22 the appointment on interface.

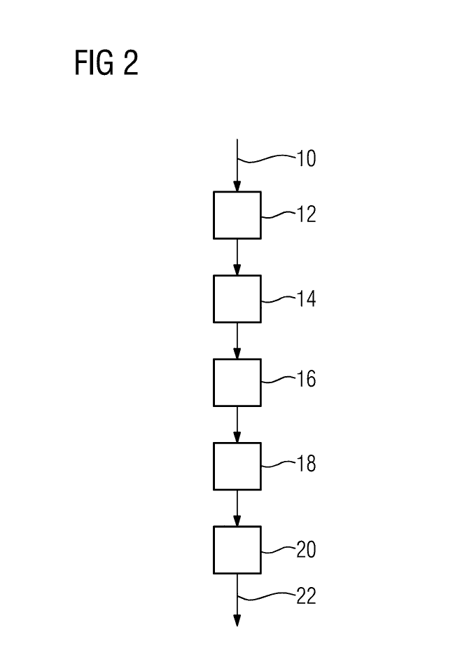

FIG. 2 shows an arrangement for the operation of a medical imaging apparatus. The medical imaging apparatus 26 is connected via the data transfer link 32 to an interface 30. The interface 30 also possesses a data transfer link 34 to the appointments scheduling system 28. With this arrangement, data can be transferred from the appointments scheduling system 28 to the medical imaging apparatus 26. A transmission of information from the medical imaging apparatus 26 directly to the appointments scheduling system 28 is also possible. The data transfer links 32 and 34 can preferably be implemented as hardwired in the form of cables or as wireless data communication links. If the appointments scheduling system 28 or the medical imaging apparatus 26 is not available due to failure of the data transfer links 32 and/or 34, the interface 30 is able to store all of the information. The information is stored on a data medium 36 or a network-attached data storage appliance 38. The data medium 36 is connected to the medical imaging apparatus 26 and the appointments scheduling system 28 and can store information from the two devices on a long-term as well as a short-term basis. The network-attached data storage appliance is connected to the medical imaging apparatus 26 and the appointments scheduling system 28. Preferably, in addition to a storage function of the network-attached data storage appliance 38, the network-attached data storage appliance 38 can also perform computational operations or execute computer programs which can be used for controlling or operating the medical imaging apparatus and/or the appointments scheduling system.

Although modifications and changes may be suggested by those skilled in the art, it is the intention of the Applicant to embody within the patent warranted hereon all changes and modifications as reasonably and properly come within the scope of the Applicant's contribution to the art.

1. A computer-implemented method for optimizing the operation of medical imaging apparatuses in one or more service centers, comprising:
a. appointment request by a patient to an appointments scheduling system;
b. acquisition of at least one patient attribute by the appointments scheduling system;
c. forwarding the patient attribute from the appointments scheduling system to the medical imaging apparatus;
d. providing a duration of a medical imaging process by the medical imaging apparatus as a function of the patient attribute, and forwarding said duration to the appointments scheduling system;
e. selection of an appointment by the appointments scheduling system as a function of the duration of the medical imaging process and an availability of the medical imaging apparatus(es); and
f. storing of the appointment selected by the patient by the appointments scheduling system.

2. The method for optimizing the operation of medical imaging apparatuses as claimed in claim 1, comprising a further step g.) of providing the selected appointment as an output via on an interface of the appointments scheduling system.

3. The method for optimizing the operation of medical imaging apparatuses as claimed in claim 1, wherein steps (c) to (f) are performed automatically.

4. The method for optimizing the operation of medical imaging apparatuses as claimed in claim 1, comprising:
determining the estimated duration of an ongoing medical imaging process during the ongoing medical imaging process;
forwarding the estimated duration of the ongoing medical imaging process to the appointments scheduling system and comparing said duration with its provided duration;
if necessary, providing a data packet containing the estimated end time; and
if necessary, adjusting the following appointments by the appointments scheduling system.

5. The method for optimizing the operation of medical imaging apparatuses as claimed in claim 1, comprising the following steps:
providing a setup time as a function of the patient attribute by the medical imaging apparatus to the appointments scheduling system; and
taking the setup time into consideration in the appointment selection by the appointments scheduling system.

6. The method for optimizing the operation of medical imaging apparatuses as claimed in claim 5, comprising selecting an appointment such that no setup times occur, by selecting successive appointment slots for medical imaging processes having the same patient attribute.

7. The method for optimizing the operation of medical imaging apparatuses as claimed in claim 1, wherein the duration of the medical imaging process is determined or calculated using averaged durations from medical imaging processes performed on the medical imaging apparatus over a period of time, preferably between one day and three months.

8. The method for optimizing the operation of medical imaging apparatuses as claimed in claim 1, wherein the patient attribute is at least one attribute selected from the group consisting of a name of the patient, a type of imaging process, a part of the body, an associated acquisition coil, an implant present in the patient, an indication, and personal details provided by the patient.

9. The method for optimizing the operation of medical imaging apparatuses as claimed in claim 1, wherein a patient declines an appointment and reports the decline to the appointments scheduling system, wherein the appointments scheduling system generates at least one further appointment by repeating step e.), wherein the patient receives the further appointment and sends the appointments scheduling system an acknowledgment, wherein step f.) is performed if the acknowledgment is positive.

10. The method for optimizing the operation of medical imaging apparatuses as claimed in claim 1, wherein the availability of a medical imaging process in step e.) is determined on the basis of multiple medical imaging apparatuses distributed over a plurality of service centers.

11. A method for operating an appointments scheduling system, comprising:
processing of an appointment request of a patient by the appointments scheduling system;
acquisition of at least one patient attribute by the appointments scheduling system;
forwarding of an appointment request to a medical imaging apparatus and/or one or more patient attributes to the medical imaging apparatus by the appointments scheduling system;
reception, by the appointments scheduling system, of a response of the medical imaging apparatus, comprising a duration of a medical imaging process and/or an acquisition coil that is installed in the medical imaging apparatus at the time of the appointment request;
calculation, by the appointments scheduling system, of the duration of an appointment on the basis of the appointment request, at least one patient attribute, the duration of the medical imaging process and, if applicable, a length of time for changing the acquisition coil; and
assignment and/or sorting of the appointments on the basis of patient attributes of the patients in order to minimize a number of acquisition coil changes.

12. A method for operating a medical imaging apparatus, comprising:
receiving an appointment request and/or at least one patient attribute;
providing a duration of a medical imaging process as a function of at least one patient attribute; and
forwarding the duration of the medical imaging process.

13. The method for operating a medical imaging apparatus as claimed in claim 12, wherein the duration of the medical imaging process is calculated as a function of a patient attribute, said duration comprising a preparation time of the medical imaging process, an acquisition time of the medical imaging process, a post-processing time of the medical imaging process, and, if applicable, a changeover time for swapping an acquisition coil of the medical imaging apparatus, wherein the duration of the medical imaging process is calculated constantly, daily, weekly, quarterly or yearly on the basis of predefined values and/or based on values determined during operation of the medical imaging apparatus and/or on manual inputs on the medical imaging apparatus.

14. An interface for minimizing setup times between an appointments scheduling system and a medical imaging apparatus,
wherein the interface is configured to forward an appointment request of the appointments scheduling system to the medical imaging apparatus;
wherein the data format can be converted by the interface;
wherein the interface is configured to record a response of the medical imaging apparatus to the appointment request and to pass on said response to the appointments scheduling system;
wherein the response is converted by the interface into a different data format; and
wherein the interface of the appointments scheduling system is configured to store the appointment request and/or the response in the event that the appointments scheduling system and/or the medical imaging apparatus cannot be reached.

15. A non-transitory, computer-readable data storage medium encoded with programming instructions, said storage medium being loaded into a scheduling computer that schedules appointments for a medical imaging apparatus, and said programming instructions causing said scheduling computer to:
receive an appointment request by a patient;
receive at least one patient attribute;
forward the patient attribute from the appointments scheduling computer to the medical imaging apparatus;
receive a duration of a medical imaging process from the medical imaging apparatus as a function of the patient attribute;
select an appointment by the appointments scheduling computer as a function of the duration of the medical imaging process and an availability of the medical imaging apparatus(es); and
store the appointment selected by the patient by the appointments scheduling computer.

16. A medical imaging apparatus comprising:
a medical data acquisition device;
a scheduling computer;
said scheduling computer being configured to receive an appointment request by a patient to an appointments scheduling computer;
said scheduling computer being configured to acquire at least one patient attribute by the appointments scheduling computer;
said scheduling computer being configured to forward the patient attribute from the appointments scheduling computer to the medical imaging apparatus;
said scheduling computer being configured to provide a duration of a medical imaging process by the medical imaging apparatus as a function of the patient attribute, and forwarding said duration to the appointments scheduling computer;
said scheduling computer being configured to select an appointment by the appointments scheduling computer as a function of the duration of the medical imaging process and an availability of the medical imaging apparatus(es); and
said scheduling computer being configured to store the appointment selected by the patient by the appointments scheduling computer.
